(19) United States
(12) Patent Application Publication
(10) Pub. No.: US 2012/0101345 A1
(43) Pub. Date: Apr. 26, 2012

(54) HEALTH MANAGEMENT TERMINAL AND THE METHOD FOR MANAGING HEALTH

(76) Inventor: SUNGWOOK PARK, Seoul (KR)

(21) Appl. No.: 12/982,589

(22) Filed: Dec. 30, 2010

(30) Foreign Application Priority Data
Oct. 20, 2010 (KR) 10-2010-0102406

(51) Int. Cl. A61B 5/00

(52) U.S. Cl. 600/300

(57) ABSTRACT

A health management terminal and method for managing health are discussed. The health management terminal includes an output module and a controller obtaining bio-signals and information regarding a time when a measurement of the bio-signals was made. The controller compares the bio-signals to a reference range, the reference range being adjusted according to the time when the measurement was made, and determines whether the bio-signals fall within the reference range. If the bio-signals are determined to fall outside of the reference range, the controller outputs, via the output module, a first user message for determining whether the obtained bio-signals are abnormal.

This application claims the benefit of the earlier filing and right of priority to Korean Application 10-2010-0102406, filed on Oct. 20, 2010, the contents of which are incorporated by reference herein in its entirety.

### BACKGROUND OF THE INVENTION

1. Field of the Invention

The present invention relates to health management and more particularly, a health management terminal and a method for obtaining bio-signals and providing a user interface for determining whether the obtained bio-signals are normal.

2. Description of Related Art

Recently, interest in health is increasing. In particular, as an interest in well-being which pursues a healthy life increases, various solutions for managing health are being developed. Related thereto, a more accurate solution capable of continuously tracking health status of a user is required

### BRIEF SUMMARY OF THE INVENTION

One object of the present invention is to provide a user interface for obtaining various bio-signals and determining whether the obtained bio-signals fall within a normal range.

Technical objects that the present invention attempts to achieve are not limited to those aspects described above. In addition, other technical objects that the present invention attempts to achieve will be clearly understood from the description provided in the following by those skilled in the art to which the present invention belongs.

A health management terminal according to one aspect of the present invention comprises an output module; and a controller obtaining bio-signals and information regarding a time when measurement of the bio-signals was made, determining whether the bio-signals fall within a reference range considering the time when the measurement was made, the reference range being changed according to the time when the measurement was made, and if the bio-signals are determined to fall outside of the reference range, outputting, via the output unit, a first user interface for determining whether the obtained bio-signals are abnormal.

A method for managing health according to one aspect of the present invention comprises obtaining bio-signals and information regarding a time when measurement of the bio-signals was made; determining whether the bio-signals fall within a reference range considering of the time when the measurement was made; and if the bio-signals are determined to fall outside of the reference range, outputting a first user interface for determining whether the obtained bio-signals are abnormal, the reference range being changed according to the time when the measurement was made.

### BRIEF DESCRIPTION OF THE DRAWINGS

The present invention will become more fully understood from the detailed description given hereinbelow and the accompanying drawings which are given by way of illustration only, and thus are not limitative of the present invention and wherein:

FIG. 1 is a simplified illustration of a system according to one embodiment of the present invention;

FIG. 2 illustrates a structure of a terminal 100 according to one embodiment of the present invention;

FIG. 3 is a flow chart illustrating a method for determining whether measured bio-signals fall within a normal range;

FIG. 4 illustrates a user interface for checking the time when measurement of bio-signals was made according to one embodiment of the present invention;

FIG. 5 illustrates a reference range according to one embodiment of the present invention;

FIG. 6 is a flow chart illustrating determination of whether bio-signals are normal according to one embodiment of the present invention and information provision according to the determination;

FIG. 7 illustrates a user interface for checking existence of factors affecting measurement of bio-signals according to one embodiment of the present invention; and

FIGS. 8 and 9 illustrate a user interface provided when bio-signals are abnormal according to one embodiment of the present invention.

### DETAILED DESCRIPTION OF THE INVENTION

The objective, characteristics, and advantages of the present invention described above will be more clearly understood by detailed description in the following related to appended drawings. In what follows, embodiments according to the present invention will be described in detail with reference to appended drawings. The same reference numbers represent the same constituting elements across the document. In addition, if specific description about functions or a structure known in the art related to the present invention is determined to unnecessarily make unclear the technical principles of the present invention, the corresponding description will be omitted.

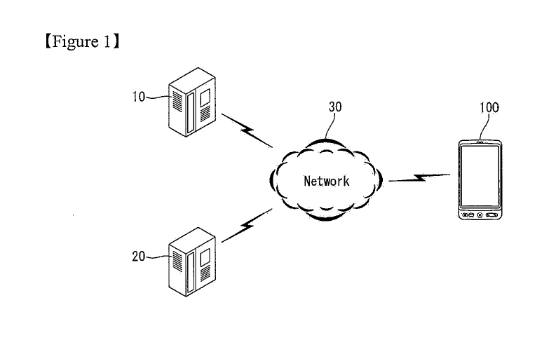

FIG. 1 is a simplified illustration of a system according to one embodiment of the present invention. A system according to one embodiment of the present invention comprises a health management server 10, a medical institution server 20, a network 30, and a health management terminal 100. The configuration above is not mandatory; therefore, more or less of the above configuration is equally allowed.

The health management server 10 can integrate and manage health-related information. Also, the health management server 10 can transmit or receive health-related information to and from at least one of the medical institution 20 and the health management terminal 100 through the network 30.

The medical institution server 20 can manage computing of a medical institution and process information received from the health management server 10 and/or the health management terminal 100. For example, the medical institution server 20 can transmit and/or receive information about reservation of a medical institution and information related to the health of a patient to and from at least one of the health management server 10 and the health management terminal 100 through the network 30.

The network 30 can provide a communication path for transmitting and receiving information by the medical institution server 20 and the health management terminal 100. For example, the network 30 can be one of a wired/wireless communication network and a mobile communication network or a combination of both.

The health management terminal 100 can provide various kinds of information related to health for the user. To this purpose, the health management terminal 100 can receive required information from the health management server 10 and the medical institution server 20 through the network 30. Specific functions of the health management terminal 100 are described in detail in what follows. Also, for the convenience of description, the health management terminal 100 is called a terminal 100 for short.

In the following, the terminal 100 will be described in more detail with reference to appended drawings. Suffixes of “module” and “unit” used for constituting elements in the following description have been introduced or used in a mixed fashion in consideration only of convenience of preparing this document; thus, the suffixes themselves do not possess respective meanings distinguishable from each other or indicate dedicated roles.

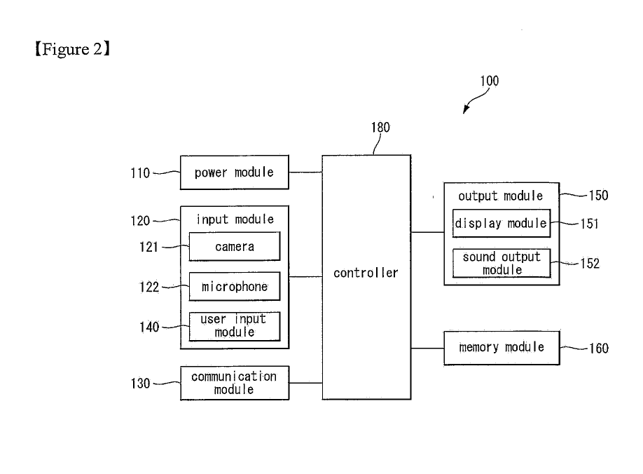

FIG. 2 illustrates a structure of a terminal 100 according to one embodiment of the present invention. The terminal 100 comprises a power module 110, an input module 120, a communication module 130, an output module 150, and a memory module 160. The configuration above is not mandatory; therefore, an electronic device which includes more or less of the above configuration can be implemented.

The power module 110 provides a power required for operating individual elements constituting the electronic device.

The input module 120 is intended for receiving audio signals, video signals, and the user input from a user; the input module 120 can include at least one of a camera 121, a microphone 122, and a user input module 140.

The camera 121 processes image frames of still images or a video obtained from an image sensor in a video communication mode or a capture mode. The processed image frame can be displayed on a display unit 151.

The image frame processed in the camera 121 can be stored in the memory module 160 or transmitted to the outside through the communication module 130. Two or more cameras can be incorporated depending on the structure of the electronic device.

The microphone 122 receives external sound signals and processes them into electrical voice data in a communication mode, a recording mode, or a voice recognition mode.

The user input module 140 can be realized by a keypad, a dome switch, a touch pad (resistive type/electrostatic type), a jog wheel, or a jog switch.

The user input module 140 applies no restriction for the realization thereof. In general, a keypad type, a wheel key type, a touch pad type, a touch screen type, and a combination of two or more of the above can be employed. Recently, a touch screen type is widely accepted in consideration of extended space utility, large size of a display screen, and a design requirement; in this case, the user input module 140 and the display module 151 to be described later can be integrated into one module.

The communication module 130 allows communication with electronic devices and can include one or more modules. The communication module 130 can utilize not only a currently existing communication means but also a communication means to be used in the future.

The output module 150 is used for generating outputs related to a visual, auditory, and tactile sense, which can include a display module 151 and a sound output module 152. The output module 150 can further include a haptic module (not shown) to generate outputs related to a tactile sense (e.g., vibration) as well as the outputs above.

The display module 151 can display information processed in the terminal 100. The display module 151 can include at least one of a liquid crystal display, a thin film transistor-liquid crystal display, an organic light-emitting diode, a flexible display, and a three dimensional display. Two or more of the display module 151 can be employed depending on the implementation type of the terminal 100.

The sound output module 152 outputs audio data received from the outside or generated in the terminal 100. In other words, the sound output module 152 can output sound signals related to the functions carried out in the terminal 100. The sound output module 152 can include a speaker, a buzzer, and the like. In addition, the sound output module 152 can output sound through an earphone jack. The user can hear output sounds by connecting his or her earphone to the earphone jack.

The memory module 160 can store a program for operating the terminal 100 and store input, output, generated data (for example, audio, still images, and video) temporarily or permanently.

The memory module 160 can include at least one type of storage media comprising of a flash memory type, a hard disk type, a multimedia card micro type, a card-type memory (for example, SD or XD memory), random access memory (RAM), static random access memory (SRAM), read-only memory (ROM), electrically erasable programmable read-only memory (EEPROM), programmable read-only memory (PROM), a magnetic memory, a magnetic disk, and an optical disk. The memory module 160 can exist as a separate module outside the terminal 100.

The controller 180 can control the overall operation of the terminal 100. For example, the controller 180, by controlling each constituting element of the terminal 100, can realize one embodiment of the present invention.

Various embodiments described in this document can be realized in a medium which is readable by a computer or a similar device through software, hardware, or a combination of both.

In terms of hardware implementation, an embodiment described in this document can be realized by at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electric units for performing functions.

As for software implementation, embodiments such as procedures and functions can be realized together with separate software modules which perform at least one function or operation. Software codes can be realized by a software application written in an appropriate program language. Also, software codes can be stored in the memory module 160 and can be executed by the controller 180.

In addition, a method described in this document can be realized by software. The software, by downloaded to the terminal 100 from a server separate from the terminal 100, can be installed in the terminal 100. For example, the terminal 100 makes a request on a particular server for software required to realize the present invention and receives the requested software by downloading; therefore, the terminal can provide the user an environment where an embodiment according to the present invention can be realized.

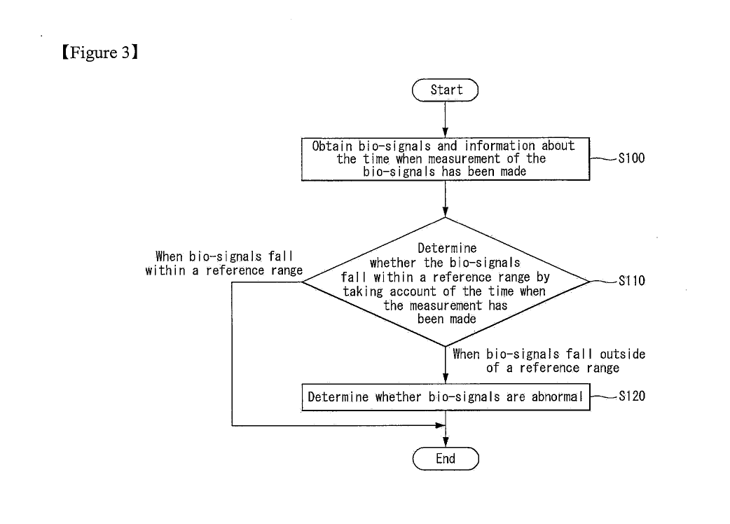

In the following, with reference to FIGS. 1 and 2, provided is a method for determining whether measured bio-signals fall within a normal range according to one embodiment of the present invention. Reference to FIGS. 1 and 2 is intended only for the convenience of description, which does not imply that the technical principles of the present invention are limited to a particular system or a particular device. In the following, with reference to FIG. 3, a method for determining whether measured bio-signals fall within a normal range according to one embodiment of the present invention will be described.

FIG. 3 is a flow chart illustrating a method for determining whether measured bio-signals fall within a normal range.

The terminal 100 obtains bio-signals and information regarding the time when measurement of the bio-signals was made S100. The terminal 100 can obtain the bio-signals and the information about the time when measurement of the bio-signals was made through S100 step.

The bio-signals comprise various health-related indicators measurable from a human body. For example, the bio-signals can include blood sugar, blood pressure, glutamic-pyruvic transaminase (GPT), cholesterol level, body temperature, and the like.

The terminal 100 can obtain the bio-signals in various ways. For example, the terminal 100, can obtain the bio-signals through a bio-signal measurement module (not shown) included in the terminal 100. Also, for example, the terminal 100 can obtain the bio-signals from other electronic devices. In other words, the terminal 100 can obtain bio-signals measured by other electronic devices through the communication module 130. By doing so, the terminal 100 can obtain quantitative values of the bio-signals.

Also, the terminal 100 can obtain information regarding the time when measurement of the bio-signals was made. The time when measurement of the bio-signals was made can be one of the time at which the bio-signals have been measured and a relative time at which the bio-signals have been measured. For example, the time at which the bio-signals have been measured can be expressed in the form of 11:00 PM. In addition, the terminal 100, if the measurement of the bio-signals was made by the terminal 100, can obtain the measurement time from a time measurement module (not shown) embedded inside the terminal 100. If the measurement of bio-signals was made by another terminal 100 rather than the terminal above, information about the time at which measurement of the bio-signals was made can be obtained from the external terminal.

Meanwhile, a relative time at which the measurement of the bio-signals was made can be expressed by the relative time with respect to various criteria affecting bio-signal values such as whether the user has had a meal, whether the user has slept, and whether the user is on an empty stomach. To be more specific, the time at which the measurement of the bio-signals was made can be classified into on an empty stomach, within two hours before a meal, within two hours after a meal, after two hours since a meal, and before sleeping. The above is only an example and the time at which measurement of the bio-signals was made can be classified by more various criteria.

The terminal 100 can obtain information regarding relative time at which measurement of the bio-signals was made in various ways. For example, the terminal 100 can receive a relative time at which measurement of bio-signals from a person whose bio-signals are measured through the input module 120. For example, the user can input through the input module 120 that the measurement of the bio-signals was made within two hours before a meal. By doing so, the terminal 100 can obtain information about a relative time at which measurement of the bio-signals was made from the user.

In another example, the terminal 100 can obtain the time at which the measurement of the bio-signals was made and based on the schedule of the user whose bio-signals are measured, estimate a relative time at which the measurement of the bio-signals was made. For example, the terminal 100 can obtain information about the time at which the measurement of the bio-signals was made. The terminal 100, as described above, can obtain information about the time at which the measurement of the bio-signals was made in different ways depending on where the bio-signals have been measured. For example, the terminal 100, if the bio-signals have been measured at the terminal 100, can obtain information about the time at which the measurement of the bio-signals was made based on the time information carried by the terminal 100. If the bio-signals have been measured in an external terminal, the terminal 100 can obtain from the external terminal information about the time at which the bio-signals have been measured.

In addition, the terminal 100 can obtain the schedule of the user whose bio-signals are measured. For example, the terminal 100 can obtain the schedule of the user from the memory module 160 or an external server. By doing so, the terminal 100 can estimate that the time of the measurement of the obtained bio-signals is 11:00AM and if it is found from the obtained schedule of the user whose bio-signals are measured that 12:30 PM is lunch time, a relative time at which the measurement of the bio-signals was made is within two hours before lunch.

Therefore, the terminal 100 can determine to which time of the user's schedule the time at which the bio-signals have been measured corresponds and, based on the result, estimate a relative time at which the measurement of the bio-signals was made.

Furthermore, the terminal 100 can output the estimated relative time at which the obtained bio-signals were measured through the output module 150. In other words, the terminal 100 can output a user interface through the output module 150 to confirm whether the information about the obtained estimated relative time is correct.

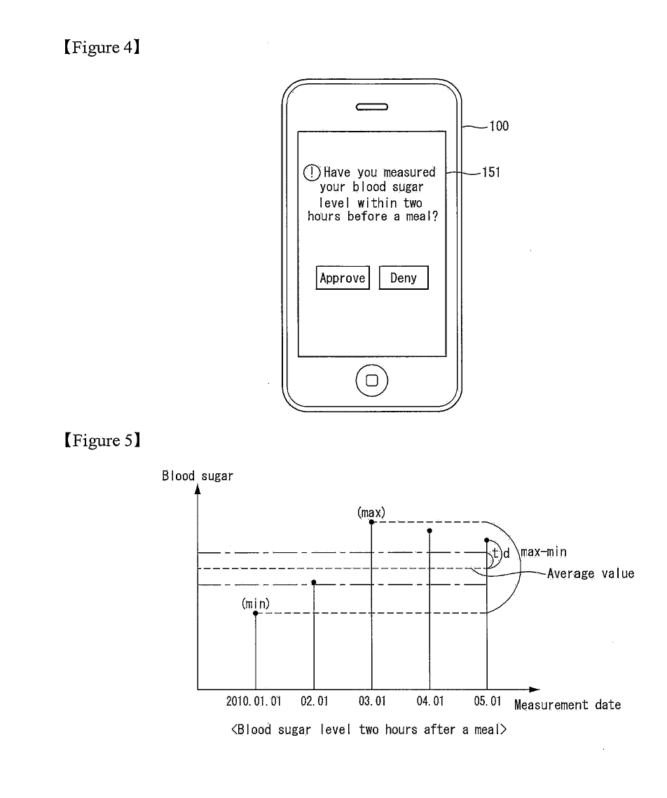

For example, the terminal 100, as shown in FIG. 4, as the estimated relative time at which the bio-signals have been measured is output, can receive a confirmation for the estimate from the user. In other words, since the terminal 100 provides a relative time at which the bio-signals have been measured, the user can enjoy the convenience where the user does not have to input a relative time at which the measurement of the bio-signals was made.

With reference to FIG. 4, if the measurement time of the bio-signals is found to be within two hours before a meal, the user can select an icon corresponding to the approval shown in FIG. 4. If the measurement time of the bio-signals is found to be more than two hours before a meal, the user can select an icon corresponding to a denial shown in FIG. 4.

If an icon corresponding to the approval is selected, the terminal can store a relative time at which the measurement was made and the obtained bio-signals in the memory module 160 to be in association with each other.

Also, if an icon corresponding to the denial is selected, the terminal 100 allows the user to directly input a relative time at which the measurement was made through the user interface module 140.

In other words, the terminal 100, by providing a user interface for receiving a relative time at which the measurement of the bio-signals was made, can receive information about the relative time at which the measurement of the bio-signals was made. According to the method describing the S100 step, the terminal 100 can obtain bio-signals and information about the time at which the measurement of the bio-signals was made.

The terminal 100 can store the obtained bio-signals and information about the time at which the measurement of the bio-signals was made to be in association with each other. For example, the terminal 100, by associating the obtained bio-signals with the information about the time at which the bio-signals have been measure, can allow considering the time at which the bio-signals have been measured at the time of analyzing the bio-signals. To be more specific, the terminal 100 can store a bio-signal A and the time at which measurement of the bio-signal A was made by associating the bio-signal A with the fact that A was measured to be of B level at the time of 11:00 PM May 1, 2010. Meanwhile, differently from the above, based on the information about a relative time at which measurement of bio-signals was made, the terminal 100 can store the bio-signal A and a relative time at which measurement of the bio-signal A was made by associating the bio-signal A with the fact that A was measured to be of B level within two hours before a meal at May 1, 2010.

The terminal 100 can associate the obtained bio-signals and the time at which the measurement of the bio-signals was made and thus store them in the memory module 160 of the terminal 100 and/or in the health management server 10.

Although the description above is given to the terminal's 100 obtaining information about a time at which the bio-signals have been measured in association with the obtained bio-signals, the terminal 100 can obtain information about various parameters affecting measurement of the bio-signals. For example, the parameters can include at least one of the parameters about the place for measuring the bio-signals, the day at which the measurement of the bio-signals was made, and the order of measurement during a day.

By taking account of the time at which the measurement was made into account, the terminal can determine whether the bio-signals fall within a reference range S110.

The terminal 100 can obtain a reference range of a bio-signal corresponding to the time at which the measurement was made. The reference range can be used as a criterion with which the measure bio-signal belongs to a proper range.

The reference range can be varied according to the time at which the measurement was made. For example, according to a relative time at which the measurement was made, which is one example of the measurement time, if the time at which the measurement was made is on an empty stomach, the reference range can be a1˜a2; if the time at which the measurement was made is within two hours before a meal, the reference range can be b1˜b2; if the time at which the measurement was made is within two hours after a meal, the reference range can be c1˜c2; if the time at which the measurement was made is after two hours since a meal, the reference range can be d1˜d2; if the time at which the measurement was made is at sleeping time, the reference range can be e1˜e2. As described above, classification about the time at which the measurement was made is only an example and can be realized in more various ways.

The terminal 100 can obtain a reference range according to the time at which the measurement was made in various ways. For example, the terminal 100 can obtain a reference range based on a standard index of a bio-signal. Also, for example, the terminal 100 can generate a reference range based on a previous record of the bio-signal. In the following, descriptions of the respective cases will be given more specifically.

In case the reference range is obtained through the standard index, the terminal 100 can obtain a reference range according to the time at which the measurement was made from at least one of the memory module 160 and an external server. For example, the external server can be at least one of a management server 10 and a medical institution server 20. In other words, in case the bio-signal is blood sugar, the terminal 100 can obtain a standard range of blood sugar corresponding to the time at which the measurement was made. To be more specific, in case the bio-signal is blood sugar and the time at which the measurement was made according to the standard range of blood sugar is on an empty stomach, the reference range can be 100 mg/dL or less. If the time at which the measurement was made is within two hours before a meal, the reference range can be 120 mg/dL or less; if the time at which the measurement was made is within two hours after a meal, 140 mg/dL or less; if before sleeping, 110 mg/dL or less.

Meanwhile, the terminal 100 can generate a reference range based on a previous record of a bio-signal measured in the past by the same user whose bio-signals are measured. In other words, the terminal 100 can classify the bio-signals measured in the past according to a relative period during which the measurement was made and based on the degree of variations of bio-signals in the past during a particular period classified according to the relative period during which the measurement was made, and generate the reference range.

For example, the terminal 100, if today is May 1, 2010; bio-signals measured in the past can be obtained, where the bio-signals have been measured at Jan. 1, 2010; Feb. 1, 2010; and Apr. 1, 2010.

The terminal 100 can classify bio-signals obtained in the past based on a relative period during which measurement was made. In other words, the terminal 100 can classify bio-signals measured at Jan. 1, 2010; Feb. 1, 2010; Mar. 1, 2010; and Apr. 1, 2010 according to a relative period during which measurement was made such as before a meal, after a meal, on an empty stomach, and at sleeping. For example, the terminal 100 can obtain information about a record of a bio-signal in the past measured within two hours before a meal at Jan. 1, 2010; Feb. 1, 2010; Mar. 1, 2010; and Apr. 1, 2010.

By doing so, the terminal 100 can obtain information about the records such as a maximum value, a minimum value, and an average value of a bio-signal measured before a meal in the past. In the same way, the terminal 100 can equally obtain information about the record of the bio-signal measured in the past by classifying the information into ‘after a meal’, ‘on an empty stomach’, and ‘at sleeping’. Furthermore, the terminal 100 can more specifically categorize the information about bio-signals measured in the past. To be more specific, the categorization can be ‘within two hours before a meal’, ‘within two hours after a meal’, and ‘two hours later after a meal’.

The terminal 100, based on the categorized information about the record of a bio-signal in the past, can obtain a reference range corresponding to a period during which at least one or more measurement was made. For example, the terminal 100 can set the maximum and the minimum value of a bio-signal in the past as a reference range. To be more specific, the terminal 100 can set the maximum and the minimum value of a bio-signal measured before meals in the past as a reference range. As shown in FIG. 5, the reference range can be between the minimum value measured at Jan. 1, 2010 and the maximum value measured at Mar. 1, 2010.

In another example, the terminal 100 can set a reference range by setting a predetermined range based on an average value of a bio-signal in the past as a variation range allowable. To be more specific, the terminal 100 can obtain an average value of a bio-signal measured before meals in the past and set a predetermined range based on the obtained average value as a reference range. As shown in FIG. 5, the reference range can be the average value from Jan. 1, 2010 to Apr. 1, 2010 with a variation of t above and below the average value. In addition, the terminal 100 can generate a reference range based on a bio-signal of the past categorized through various algorithms.

If multiple reference ranges are generated by different algorithms from each other for a particular period during which measurements have been made, the terminal 100 can receive a selection signal to select one reference range among multiple reference ranges from the user. In other words, the user can select one of the multiple reference ranges generated according to the bio-signal records in the past through the user input module 140.

The terminal 100, if multiple reference ranges are generated by the same algorithm for a particular period during which measurements have been made, can select a reference range which was generated most recently.

The terminal 100 can generate a reference range based on a bio-signal record of the past by itself, the terminal 100 can obtain a reference range based on a bio-signal record of the past from an external server through the communication module 130.

In addition, if the terminal 100 obtains a bio-signal measurement time which is an example of a time at which measurement of bio-signals was made in the S100 step, a reference range corresponding to the measurement time of bio-signals can be generated in the same way as the method for generating a reference range described above.

By doing so, the terminal 100 can obtain a reference range of the bio-signal corresponding to each of the measurement period which measurement was made.

The terminal 100 can determine whether the bio-signal falls within the obtained reference range. The terminal 100 can consider the time at which the measurement was made when comparing the bio-signal with the obtained reference range. In other words, the terminal 100 can compare the bio-signal obtained in the S100 step with the time at which measurement of the obtained bio-signal was made. In the following, it is assumed that the time at which measurement of a bio-signal was made is a relative time.

For example, the terminal 100 can obtain the information that the time at which blood sugar level as a bio-signal and measurement thereof was obtained in the S100 step is two hours after a meal. The terminal 100 can obtain a reference range corresponding to two hours after a meal at which measurement of blood sugar was made. As described above, a reference range of blood sugar two hours after a meal can be obtained from a standard index or from information about bio-records in the past. In this way, the terminal 100 can compare a reference range belonging to the same period as blood sugar measured in the S100 step.

More specifically, if the reference range was generated based on the information about bio-records in the past, as shown in FIG. 5, the terminal 100 determines whether blood sugar measured two hours after meals at May 1 falls within the reference range.

At this time, if the reference range is determined by intermediate values lying between the maximum value and the minimum value of a bio-signal in the past, as shown in FIG. 5, since the bio-signal measured at May 1 lies between the measurement at Mar. 1 which is the maximum of the bio-signal in the past and the measurement at Jan. 1 which is the minimum thereof, the terminal 100 can determine that the blood sugar level measured at May 1 falls within the reference range (max-min).

Differently from the above, if the reference range is determined as the range of maximum/minimum t with respect to the average value of a bio-signal in the past, as shown in FIG. 5, since the bio-signal measured at May 1 has a magnitude of d which is larger than t in terms of the average value of the bio-signal in the past, the terminal 100 determines that the blood sugar level measured at May 1 falls outside of the reference range (average value±t).

If the measured bio-signal is determined to fall within the reference range according to the determination, the terminal can terminate the corresponding procedure. If the measure bio-signal is determined to fall outside the reference range, the terminal 100 can determine whether the bio-signal is normal or not S120. the bio-signal is outside the reference range, the terminal 100 can provide a user interface for determining whether the bio-signal is normal or not.

For example, the terminal 100 can provide the user interface for receiving an input to know whether any factors exist affecting the measurement of the bio-signals.

The factors affecting the measurement of the bio-signals can be various factors affecting the measured bio-signals. For example, if the bio-signal is blood sugar, factors affecting the bio-signal can be physical exercise, nutrition, and the like. In other words, even if the time at which measurement of the blood sugar was made falls within two hours before a meal in the schedule of the user, if the user takes food, measured blood sugar level can be increased. In this case, if the measured blood sugar level is compared with a reference range corresponding to two hours before a meal, the measured blood sugar level can fall outside the reference range due to the factor of taking food. In this case, however, since the bio-signal cannot be determined to be abnormal, it is necessary to check from the user whose bio-signals are measured whether there are factors affecting the measured bio-signals.

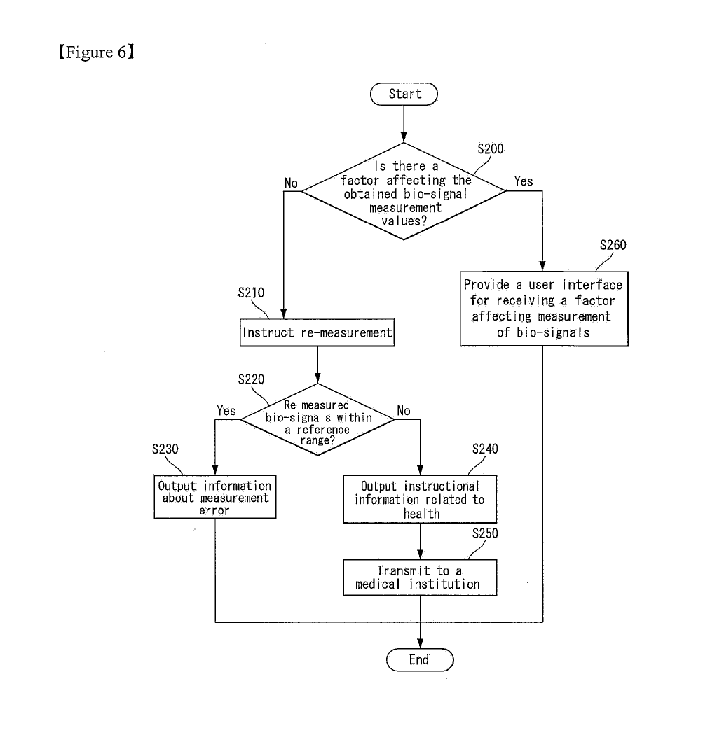

In the following, with reference to FIG. 6, the S120 step will be described more specifically. FIG. 6 is a flow chart illustrating determination of whether bio-signals are normal according to one embodiment of the present invention and information provision according to the determination.

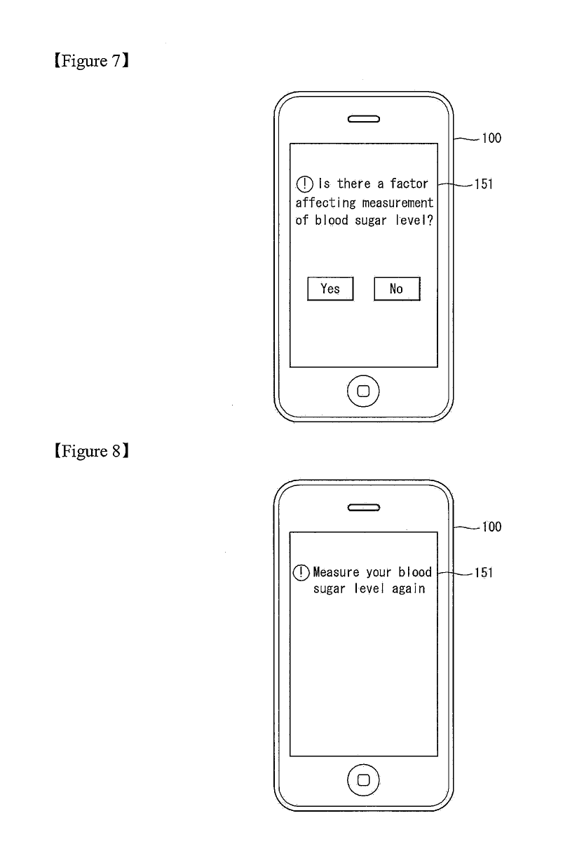

The terminal 100 can check whether there are factors affecting the obtained bio-signal measurement values S200. For example, as shown in FIG. 7, the terminal 100 can provide a user interface for checking whether there are factors affecting measurement of bio-signals through the output module 150.

Although not shown in FIG. 7, by including an example of a factor affecting the measurement of the bio-signals, the user interface for checking whether there are factors affecting the measurement of bio-signals can help the user determine and select. In other words, the user whose bio-signals are measured can prevent missing even the factors regarded as not affecting the bio-signal values.

If there are no factors affecting the bio-signal measurement values obtained from the user, the terminal 100 can output an interface indicating re-measurement of the bio-signals 5210. In other words, the terminal 100, receiving a signal from the user through the user input module 140 that there are no factors affecting the bio-signal measurement values, as shown in FIG. 8, can output information indicating re-measurement of the bio-signals through the output module 150.

The terminal 100 can determine whether the re-measured bio-signals fall within a reference range S220. The terminal 100 can obtain the re-measured bio-signal and information about the period during which the bio-signal was re-measured; and obtain a reference range corresponding to the period during which the bio-signals have been re-measured. The terminal 100 can then determine whether the re-measured bio-signals fall within the reference range corresponding to the period during which the bio-signals have been re-measured.

The terminal 100 can output information about measurement error if the re-measured bio-signals fall within a reference range S230.

In this case, the terminal 100 can determine the case above to be normal even though the measured bio-signals have temporarily fallen outside the reference range. The terminal 100 can output through the output module 150 the fact that an error has occurred in the bio-signals measured first due to a certain cause. Therefore, the terminal 100 can prevent a malfunction by which a bio-signal can be determined to be abnormal due to a device error.

In addition, when the re-measured bio-signals are determined to fall within a reference range, the terminal 100 can make sure the normality of the bio-signals by recommending again a third measurement.

The terminal 100, if the re-measured bio-signals are determined to fall outside a reference range, can output instructional information related to health S240. For example, if the re-measured bio-signals are also determined to fall outside a reference range, the terminal 100 can determine the bio-signals to be abnormal. In this case, the terminal 100 can output through the output module instructional information related to health prepared beforehand depending on the degree of abnormality of the bio-signals. More specifically, the instructional information related to health denotes the information related to first aid measures which the user whose bio-signals are measured can take for his or her health management for the present. The information related to first aid measures can be varied including which medicine to take and application for hospital admittance, for example. At this time, if the information indicates application for hospital admittance, the terminal 100 can provide the user whose bio-signals are measure with an interface for reserving a medical treatment by connecting to the designated hospital.

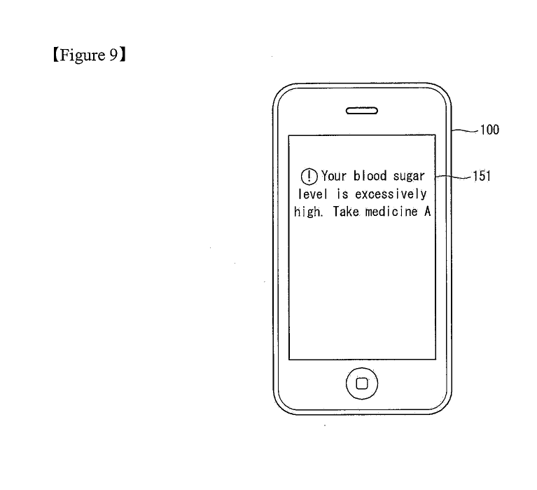

As shown in FIG. 9, the terminal 100 can output through the output module 150 information indicating taking a medicine A together with information that current blood sugar level is high.

For example, the terminal 100 can obtain information about first aid measures in case of high blood sugar level from an external server and based on the obtained information about first aid measures, generate instructional information related to health and output the generated information. Accordingly, the user whose bio-signals are measured can immediately take proper measures according to his or her health status. Also, if it is determined that the re-measured bio-signals fall outside a reference range, the terminal 100 can transmit information related to the bio-signals to a medical institution.

The terminal 100, by transmitting at least one of bio-signals measured first, re-measured bio-signals, and information about periods during which measurements of the corresponding bio-signals have been made to the medical institution server 30, can enable a doctor to consider the transmitted data as baseline data. Meanwhile, if the terminal 100 receives an input indicating that there exists a factor affecting the obtained bio-signal measurement values, the terminal 100 can output a user interface for receiving a factor affecting the measurement of bio-signals S260. The terminal 100, by receiving a factor affecting the measurement of the bio-signals, can construct a database reflecting how much the factor affects the bio-signal values. In this case, the terminal 100, even if the bio-signals are determined to fall outside a reference range at the S110 step, can postpone determining whether the bio-signals are normal or abnormal.

In other words, the terminal 100 re-determines whether the bio-signals fall within a reference range based on bio-signals obtained afterwards and information about a period during which the measurement of the bio-signals was made; and based on the determination result, can determine whether the bio-signals are normal.

According to the description above, the terminal 100 can perform S120 step of FIG. 3 with reference to FIG. 6 as described below. With reference to FIG. 3, according to one embodiment of the present invention, the terminal 100 can first determine whether obtained bio-signals are normal based on a reference range and secondly, if the obtained bio-signals are determined to fall outside the reference range, determine whether the obtained bio-signals are normal by taking the factors which affected bio-signal measurement into consideration. Therefore, the terminal 100 can minimize an error which may occur while determining whether bio-signals are normal.

In the description above for describing embodiments of the present invention, bio-signals and a period during which measurement of the bio-signals was made have been obtained; and whether the bio-signals fall within a reference range was determined by comparing the reference range corresponding to the period during which the measurement of the bio-signals was made with the bio-signals. However, in comparing the bio-signals and the reference range, at least one of information about the place for measuring the bio-signals, information about the day at which the measurement of the bio-signals was made, and information about when the measurement of the bio-signals was made in the order of measurement during a day can be taken into account. In other words, if some bio-signals have been measured at a place A, the reference range compared with the bio-signals may have been generated based on the bio-signals measured in the past at the place of A. Also, if some bio-signals have been measured on Saturday, the reference range compared with the bio-signals may have been generated based on the bio-signals measured in the past Saturday. By doing so, relationship between measured bio-signals and a reference range can be more strengthened.

Various embodiments described in this document can be performed individually or in combination with each other. Also, steps constituting an embodiment can be performed in combination with the steps constituting different embodiments.

A method according to the present invention described above can be provided being recorded in a non-transitory computer-readable medium.

A computer-readable medium includes all kinds of recording devices in which data that can be read out by a computer system are stored. Examples of a non-transitory computer-readable recording device include ROM, RAM, CD-ROM, DVD±ROM, DVD-RAM, a magnetic tape, a floppy disk, a hard disk, and an optical data storage device. In addition, a computer-readable recording medium can be distributed across computing apparatus connected by a network, where computer-readable codes can be stored and executed in a distributed way.

Also, a method according to the present invention can be performed by software. When the method is performed by software, constituting means of the present invention are code segments which perform necessary tasks. Programs or code segments can be stored in a medium which can be recognized by a processor or can be transmitted by computer data signals combined with a carrier wave in a transmitting medium or in a communication network.

According to the present invention, by providing a user interface for determining existence of factors affecting measurement of bio-signals, a health management terminal and a method for managing health can be provided which can determine more accurately whether bio-signals are normal.

Advantageous effects due to the present invention are not limited to those described above but can include various other effects which can be derived from what are described.

Since the present invention described above can be substituted, modified, and changed in various ways by those skilled in the art to which the present invention belongs without departing the technical principles of the present invention, the present invention is not limited to the embodiments described above and appended drawings. In addition, embodiments described in this document are not intended to limited applications but the whole of the embodiments or part thereof can be selectively combined to allow various modifications of the embodiments

What is claimed is:

1. A health management terminal, comprising:
an output module; and
a controller obtaining bio-signals and information regarding a time when a measurement of the bio-signals was made, comparing the bio-signals to a reference range, the reference range being adjusted according to the time when the measurement was made, determining whether the bio-signals fall within the reference range, and, if the bio-signals are determined to fall outside of the reference range, outputting, via the output module, a first user message for determining whether the obtained bio-signals are abnormal.

2. The terminal of claim 1, wherein the time when the measurement was made is a relative time about at least one of a mealtime, a sleep time, and an amount of time on an empty stomach.

3. The terminal of claim 2, wherein the controller estimates the relative time and outputs a second user message for confirming the estimated relative time through the output module.

4. The terminal of claim 3, wherein the controller obtains time information about at least one of mealtime, sleep time, and amount of time on an empty stomach from a schedule and estimates the relative time based on the obtained time information.

5. The terminal of claim 3, wherein if the estimated relative time is confirmed, the controller stores the obtained bio-signals and the estimated relative time in association with each other.

6. The terminal of claim 3, wherein if an input indicating that the estimated relative time is inaccurate is received, the controller outputs a third user message for receiving the relative time through the output module.

7. The terminal of claim 1, wherein the reference range is generated based on previous bio-signals at the same relative time by the same user who measured the obtained bio-signals.

8. The terminal of claim 2, wherein the reference range is a recommended standard bio-signal range with respect to the relative time.

9. The terminal of claim 1, wherein the controller receives an input about whether there is a factor affecting the bio-signal measurement.

10. The terminal of claim 9, wherein if the input indicating that there is no factor affecting the bio-signal measurement is received, the controller instructs re-measurement of the bio-signals.

11. The terminal of claim 10, wherein if a new bio-signal obtained according to the re-measurement instruction is determined to fall outside a reference range corresponding to the time when measurement of the new bio-signal was made, the controller outputs instructional information related to health through the output module.

12. The terminal of claim 10, wherein if a new bio-signal obtained according to the re-measurement instruction is determined to fall outside of a reference range corresponding to the time when measurement of the new bio-signal was made, the controller transmits the newly measured bio-signal and information about the time when measurement of the new bio-signal was made to a medical institution.

13. The terminal of claim 9, wherein if the input indicating that there is a factor affecting measurement of the bio-signals, the controller receives information related to factors affecting the bio-signal measurement through the output module.

14. A method for managing health, comprising:
obtaining bio-signals and information about a time when measurement of the bio-signals was made;
comparing the bio-signals to a reference range adjusted for the time when the measurement was made;
determining whether the bio-signals fall within the reference range; and
outputting a first user message for determining whether the obtained bio-signals are abnormal if the bio-signals are determined to fall outside of the reference range.

15. The method of claim 14, further comprising calculating time the measurement was based on a schedule; and
outputting a second user message for confirming the estimated relative time.

16. The method of claim 14, further comprising generating the reference range based on previous bio-signals made at the same time and by the same user.

17. The method of claim 14, further comprising receiving an input about whether there is a factor affecting the bio-signal measurement.

18. The method of claim 17, further comprising instructing re-measurement of the bio-signals if the input indicating that there is no factor affecting the bio-signal measurement is received through the first user interface.

19. The method of claim 17, further comprising outputting a fourth user message for receiving information related to factors affecting the bio-signal measurement if the input indicating that there is a factor affecting measurement of the bio-signals through the first user interface.

20. A non-transitory computer-readable recording medium recording programs for performing a method of claim 14 in a computer
